(19) United States
(12) Patent Application Publication
(10) Pub. No.: US 2021/0290481 A1
(43) Pub. Date: Sep. 23, 2021

(54) SYSTEM, METHOD AND APPARATUS FOR HELPING A MAN GAIN HIS MOST MULTIPLE ORGASMS IN A SINGLE SESSION

(71) Applicant: Wison Hau, Dublin, CA (US)

(72) Inventor: Wison Hau, Dublin, CA (US)

(21) Appl. No.: 16/826,222

(22) Filed: Mar. 21, 2020

(51) Int. Cl. A61H 19/00; G06F 3/01

(52) U.S. Cl. CPC A61H 19/32; G06F 3/011; A61H 2201/5007; A61H 2205/087; A61H 2201/5048; A61H 2201/5058; A61H 2201/5043

(57) ABSTRACT

System, method and apparatus stimulating a penis to keep gaining multiple orgasms for more than 1 hours without any refractory periods are provided. The system comprises at least one smart vagina including at least one bio-signal measuring sensor acquiring bio-signal data from the penis, at least one controller processing the bio-signal data to compute at least one penis activity measurement and at least one actuator controlled or instructed by the at least one controller to massage or stimulate the penis to achieve multiple orgasms.

### TECHNICAL FIELD

The present invention relates generally to male multiple orgasms and, in particular, to an innovative smart vaginal so as to help a male gain more than 10 thousand orgasms in just one single session.

### BACKGROUND ART

In a study of almost 32,000 men published in the journal of European Urology, researchers found high levels of sexual activity can reduce the risk of contracting prostate cancer by 33 percent.

A 1997 study of villagers in the town of Caerphilly, South Wales found that men having the most orgasms (two a week) were 50 percent less likely to die than those having the fewest (one a month).

More Orgasms means more for men. Unfortunately, a man only gains less than one-thousandth of the total orgasms which he should have in his life. Men always think more than 100 orgasms in one session are impossible. The reason is that a man does not understand his penis well so that he cannot perfectly stimulate his penis in his sexual activity.

Therefore, a need exists for a system, method and apparatus for reliably helping a man perfectly stimulate his penis in his sexual activity so that the man can achieve more than 10 thousand orgasms in only one session.

### SUMMARY OF THE INVENTION

A system comprises at least one smart vagina including at least one bio-signal measuring sensor acquiring bio-signal data from the penis, at least one controller processing the bio-signal data to compute at least one penis activity measurement and at least one actuator controlled or instructed by the at least one controller to massage or stimulate the penis to achieve multiple orgasms; and means for determining, using said at least one penis activity measurement, the difference between said penis current status and said penis orgasm status, in accordance with said determined difference, determining a stimulation pattern said penis reaches multiple orgasms or is in a continuously orgasmic state, and controlling or instructing said at least one actuator to massage said penis per said stimulation pattern. A method comprises starting a multiple orgasms session for a man per his instruction and stimulating said man's penis to achieve an erection; continuously acquiring bio-signal data from said penis using said at least one bio-signal measuring sensor during the session; processing said bio-signal data to compute at least one penis activity measurement; determining the difference between said penis current status and said penis orgasm status using said at least one penis activity measurement; in accordance with said determined difference, determining the stimulation pattern said penis reaches multiple orgasms or is in a continuously orgasmic state; in accordance with said stimulation pattern, controlling or instructing said at least one actuator to massage said penis so that said penis keeps gaining multiple orgasms without any refractory periods during the session. An apparatus comprises at least one smart vagina holding the penis of a man or inserted by the penis of a man, wherein said at least one smart vagina including means for automatically reshaping said at least one smart vagina to said penis favorite structure and tightness based on the current shape and stiffness of said penis; at least one bio-signal measuring sensor acquiring bio-signal data from said penis; at least one controller processing said bio-signal data to compute at least one penis activity measurement and determine, using said at least one penis activity measurement, a stimulation pattern said penis reaches multiple orgasms or is in a continuously orgasmic state; and at least one actuator, using said stimulation pattern, controlled or instructed by said at least one controller to massage or stimulate said penis so that said man can achieve the first orgasm and then gain more than 10 thousand orgasms in just one session.

### DETAILED DESCRIPTION OF A PREFERRED EMBODIMENT

Generally, the present invention encompasses a system for helping a man gain his most multiple orgasms in a single session comprising: At least one smart vagina holding the penis of a man or inserted by the penis of a man, wherein said at least one smart vagina including at least one bio-signal measuring sensor acquiring bio-signal data from said penis, at least one controller processing said bio-signal data to compute at least one penis activity measurement, and at least one actuator controlled or instructed by said at least one controller to massage or stimulate said penis so that said man can achieve the first orgasm and then gain more than 10 thousand orgasms in said single session; and means for determining, using said at least one penis activity measurement, the difference between said penis current status and said penis orgasm status, in accordance with said determined difference, determining a stimulation pattern said penis reaches multiple orgasms or is in a continuously orgasmic state, and controlling or instructing said at least one actuator to massage said penis per said stimulation pattern.

The system further includes at least one display; at least one speaker; at least one sensor acquiring bio-signal data from said man; and means for determining, wherein using said bio-signal data, said penis favorite videos and sounds, collecting and storing said videos and sounds, and playing said videos and sounds to stimulate said penis.

The system further includes at least one semen collector; at least one semen storage array storing all-time semen ejaculated by said penis; means for collecting semen ejaculated by said penis, analyzing said semen and generating health report, and sealing said semen stamped with date and time to a semen storage array automatically; and means for generating health-enhancing recipes for said man based on the generated health report and providing or delivering health-enhancing foods related to said recipes to said man per his request.

The system further includes at least one smart vaginal opening, the entrance of said penis, connected to said at least one smart vagina; means for reshaping the shape of said at least one smart vaginal opening, wherein the shape including but not limited to a simulated virgin vaginal opening shape, a simulated less sexual experienced vaginal opening shape, a simulated more sexual experienced vaginal opening shape, said penis favorite vaginal opening shape, or the innovative vaginal opening shape stimulating said penis the most; and means for automatically reshaping said at least one smart vaginal opening to said penis favorite structure and tightness based on the current shape and stiffness of said penis.

The at least one smart vagina further includes means for reshaping the shape of said at least one smart vagina, wherein the shape including but not limited to a simulated virgin vagina, a simulated less sexual experienced vagina, a simulated more sexual experienced vagina, said penis favorite vagina, or the innovative vagina stimulating said penis the most; and means for automatically reshaping said at least one smart vagina to said penis favorite structure and tightness based on the current shape and stiffness of said penis.

The system further includes means for timestamping every orgasm of said penis, computing and recording the time interval between adjacent orgasms of said penis, and displaying the cumulative number of the orgasms said penis gained in said session.

The stimulation pattern may be described by parameters: the stimulated area of said penis and its surroundings, massage mode for said stimulated area, stimulation intensity of said massage mode, and stimulation frequency of said massage mode.

The system further includes Virtual Reality (VR); and means for providing said man his favorite intercourse scene by said Virtual Reality (VR).

The single session means said penis stimulation process starting from the first orgasm of said penis and ending at the beginning of said penis refractory period.

Said at least one actuator comprises said man with said penis

Generally, the present invention encompasses a method for helping a man gain his most multiple orgasms in a single session, performed by a smart vaginal comprising at least one bio-signal measuring sensor and at least one actuator, comprising: starting a multiple orgasms session for a man per his instruction and stimulating said man's penis to achieve an erection; continuously acquiring bio-signal data from said penis using said at least one bio-signal measuring sensor during the session; processing said bio-signal data to compute at least one penis activity measurement; determining the difference between said penis current status and said penis orgasm status using said at least one penis activity measurement; in accordance with said determined difference, determining the stimulation pattern said penis reaches multiple orgasms or is in a continuously orgasmic state; in accordance with said stimulation pattern, controlling or instructing said at least one actuator to massage said penis so that said penis keeps gaining multiple orgasms without any refractory periods during the session.

The method further includes connecting to the Internet and using Internet resources to provide penile health services including, but not limited to, penile health indicators related to said penis; and authenticating said man with penis recognition or face recognition.

Generally, the present invention encompasses a apparatus for helping a man gain his most multiple orgasms comprising: at least one smart vagina holding the penis of a man or inserted by the penis of a man, wherein said at least one smart vagina including means for automatically reshaping said at least one smart vagina to said penis favorite structure and tightness based on the current shape and stiffness of said penis; at least one bio-signal measuring sensor acquiring bio-signal data from said penis; at least one controller processing said bio-signal data to compute at least one penis activity measurement and determine, using said at least one penis activity measurement, a stimulation pattern said penis reaches multiple orgasms or is in a continuously orgasmic state; and at least one actuator, using said stimulation pattern, controlled or instructed by said at least one controller to massage or stimulate said penis so that said man can achieve the first orgasm and then gain more than 10 thousand orgasms in just one session.

The apparatus further includes Virtual Reality (VR); and means for providing said man his favorite intercourse scene by said Virtual Reality (VR).

The apparatus further comprises means for timestamping every orgasm of said penis, computing and recording the time interval between adjacent orgasms of said penis, and displaying the cumulative number of the orgasms said penis gained in said session.

The apparatus further comprises at least one display; at least one speaker; at least one sensor acquiring bio-signal data from said man; and means for determining, wherein using the bio-signal data, said penis favorite videos and sounds, collecting and storing said videos and sounds, and playing said videos and sounds to stimulate said penis.

A preferred embodiment is that a robot with at least one smart vaginal which has bio-signal measuring sensors acquiring bio-signal data from the penis of a user and actuators stimulating and massaging the penis. A man instructs the robot to start a multiple orgasms session for him; the robot stimulates the man's penis to achieve an erection; said robot continuously acquires bio-signal data from the penis using its bio-signal measuring sensors; said robot processes the bio-signal data to compute the penis activity measurements; said robot determines the difference between the penis current status and the penis orgasm status using the penis activity measurements; in accordance with the determined difference, said robot computers and determines a stimulation pattern that helps the penis reach multiple orgasms or be in a continuously orgasmic state; in accordance with the stimulation pattern, instructs the actuators to massage the penis so that the penis keeps gaining multiple orgasms without a refractory period until said robot detects that the penis needs to end the session.

In the foregoing specification, the present invention has been described with reference to specific embodiments. However, one of ordinary skill in the art will appreciate that various modifications and changes may be made without departing from the scope of the present invention as set forth in the appended claims. Accordingly, the specification is to be regarded in an illustrative rather than a restrictive sense, and all such modifications are intended to be included within the scope of the present invention.

Benefits, other advantages, and solutions to problems have been described above with regard to specific embodiments of the present invention. However, the benefits, advantages, solutions to problems, and any element(s) that may cause or result in such benefits, advantages, or solutions, or cause such benefits, advantages, or solutions to become more pronounced are not to be construed as a critical, required, or essential feature or element of any or all the claims. As used herein and in the appended claims, the term “comprising,” or any other variation thereof is intended to refer to a non-exclusive inclusion, such that a process, method, apparatus, or system that comprises a list of elements does not include only those elements in the list, but may include other elements not expressly listed or inherent to such process, method, apparatus, or system.

1. A system for helping a man gain his most multiple orgasms in a single session comprising:
at least one smart vagina holding the penis of a man or inserted by the penis of a man, wherein said at least one smart vagina including at least one bio-signal measuring sensor acquiring bio-signal data from said penis, at least one controller processing said bio-signal data to compute at least one penis activity measurement, and at least one actuator controlled or instructed by said at least one controller to massage or stimulate said penis so that said man can achieve the first orgasm and then gain more than 10 thousand orgasms in said single session; and
means for determining, using said at least one penis activity measurement, the difference between said penis current status and said penis orgasm status, in accordance with said determined difference, determining a stimulation pattern said penis reaches multiple orgasms or is in a continuously orgasmic state, and controlling or instructing said at least one actuator to massage said penis per said stimulation pattern.

2. The system of claim 1 further comprising:
at least one display;
at least one speaker;
at least one sensor acquiring bio-signal data from said man; and
means for determining, wherein using said bio-signal data, said penis favorite videos and sounds, collecting and storing said videos and sounds, and playing said videos and sounds to stimulate said penis.

3. The system of claim 1 further comprising:
at least one semen collector;
at least one semen storage array storing all-time semen ejaculated by said penis;
means for collecting semen ejaculated by said penis, analyzing said semen and generating health report, and sealing said semen stamped with date and time to a semen storage array automatically; and
means for generating health-enhancing recipes for said man based on the generated health report and providing or delivering health-enhancing foods related to said recipes to said man per his request.

4. The system of claim 1 further comprising:
at least one smart vaginal opening, the entrance of said penis, connected to said at least one smart vagina;
means for reshaping the shape of said at least one smart vaginal opening, wherein the shape including but not limited to a simulated virgin vaginal opening shape, a simulated less sexual experienced vaginal opening shape, a simulated more sexual experienced vaginal opening shape, said penis favorite vaginal opening shape, or the innovative vaginal opening shape stimulating said penis the most; and
means for automatically reshaping said at least one smart vaginal opening to said penis favorite structure and tightness based on the current shape and stiffness of said penis.

5. The system of claim 1, wherein said at least one smart vagina further comprising:
means for reshaping the shape of said at least one smart vagina, wherein the shape including but not limited to a simulated virgin vagina, a simulated less sexual experienced vagina, a simulated more sexual experienced vagina, said penis favorite vagina, or the innovative vagina stimulating said penis the most; and
means for automatically reshaping said at least one smart vagina to said penis favorite structure and tightness based on the current shape and stiffness of said penis.

6. The system of claim 1 further comprising:
means for timestamping every orgasm of said penis, computing and recording the time interval between adjacent orgasms of said penis, and displaying the cumulative number of the orgasms said penis gained in said session.

7. The system of claim 1, wherein the stimulation pattern described by parameters: the stimulated area of said penis and its surroundings, massage mode for said stimulated area, stimulation intensity of said massage mode, and stimulation frequency of said massage mode.

8. The system of claim 1 further comprising:
Virtual Reality (VR); and
means for providing said man his favorite intercourse scene by said Virtual Reality (VR).

9. The system of claim 1, wherein the single session means said penis stimulation process starting from the first orgasm of said penis and ending at the beginning of said penis refractory period.

10. The system of claim 1, wherein said at least one actuator comprising said man with said penis.

11. A method for helping a man gain his most multiple orgasms in a single session, performed by a smart vaginal comprising at least one bio-signal measuring sensor and at least one actuator, comprising:
starting a multiple orgasms session for a man per his instruction and stimulating said man's penis to achieve an erection;
continuously acquiring bio-signal data from said penis using said at least one bio-signal measuring sensor during the session;
processing said bio-signal data to compute at least one penis activity measurement;
determining the difference between said penis current status and said penis orgasm status using said at least one penis activity measurement;
in accordance with said determined difference, determining the stimulation pattern said penis reaches multiple orgasms or is in a continuously orgasmic state;
in accordance with said stimulation pattern, controlling or instructing said at least one actuator to massage said penis so that said penis keeps gaining multiple orgasms without any refractory periods during the session.

12. The method of claim 11 further comprising:
connecting to the Internet and using Internet resources to provide penile health services including, but not limited to, penile health indicators related to said penis; and
authenticating said man with penis recognition or face recognition.

13. An apparatus for helping a man gain his most multiple orgasms comprising:
at least one smart vagina holding the penis of a man or inserted by the penis of a man, wherein said at least one smart vagina including means for automatically reshaping said at least one smart vagina to said penis favorite structure and tightness based on the current shape and stiffness of said penis;
at least one bio-signal measuring sensor acquiring bio-signal data from said penis;
at least one controller processing said bio-signal data to compute at least one penis activity measurement and determine, using said at least one penis activity measurement, a stimulation pattern said penis reaches multiple orgasms or is in a continuously orgasmic state; and
at least one actuator, using said stimulation pattern, controlled or instructed by said at least one controller to massage or stimulate said penis so that said man can achieve the first orgasm and then gain more than 10 thousand orgasms in just one session.

14. The apparatus of claim 13 further comprising:
Virtual Reality (VR); and
means for providing said man his favorite intercourse scene by said Virtual Reality (VR).

15. The apparatus of claim 13 further comprising:
means for timestamping every orgasm of said penis, computing and recording the time interval between adjacent orgasms of said penis, and displaying the cumulative number of the orgasms said penis gained in said session.

16. The apparatus of claim 13 further comprising:
at least one display;
at least one speaker;
at least one sensor acquiring bio-signal data from said man; and
means for determining, wherein using the bio-signal data, said penis favorite videos and sounds, collecting and storing said videos and sounds, and playing said videos and sounds to stimulate said penis.
